(19) United States
(12) Patent Application Publication
(10) Pub. No.: US 2026/0124047 A1
(43) Pub. Date: May 7, 2026

(54) ANTENNA FEEDTHROUGHS FOR BODILY IMPLANTS

(71) Applicant: Boston Scientific Scimed, Inc., Maple Grove, MN (US)

(72) Inventors: Trey Henry Achterhoff, Arden Hills, MN (US); Kevin James Ivanca, Minneapolis, MN (US); Jason Lahr, Coon Rapids, MN (US); Matthew William Couri, Plymouth, MN (US); Hannah Mitchell, Minneapolis, MN (US)

(21) Appl. No.: 19/377,260

(22) Filed: Nov. 3, 2025

Related U.S. Application Data
(60) Provisional application No. 63/717,621, filed on Nov. 7, 2024.

(51) Int. Cl. A61F 2/48; A61F 2/26

(52) U.S. Cl. CPC A61F 2/482; A61F 2/26; A61F 2/484

(57) ABSTRACT

A device includes a bodily implant having a housing defining a cavity. The housing has a top portion defining a first opening, a second opening, and a third opening. The device includes an electronic control system that is at least partially disposed within the cavity. The device includes a fluid control system having a pump. The fluid control system is at least partially disposed within the cavity. The device includes a recharge antenna having a first portion extending through the first opening of the top portion of the housing and into the cavity defined by the housing. The recharge antenna has a second portion extending through the second opening of the top portion of the housing and into the cavity defined by the housing. The device includes a communications antenna extending through the third opening of the top portion of the housing and into the cavity defined by the housing.

### CROSS-REFERENCE TO RELATED APPLICATION

This application claims priority to U.S. Provisional Patent Application No. 63/717,621, filed on Nov. 7, 2024, entitled “ANTENNA FEEDTHROUGHS FOR BODILY IMPLANTS”, the disclosure of which is incorporated by reference herein in its entirety.

### TECHNICAL FIELD

This disclosure relates generally to bodily implants, and more specifically to bodily implants that include housings that contain electrical components and antennas.

### BACKGROUND

Active implantable fluid-operated inflatable devices can include one or more pumps that regulate the flow of fluid between different portions of the implantable device. One or more valves can be positioned within fluid passageways of the device to direct and control the flow of fluid to achieve inflation, deflation, pressurization, depressurization, activation, deactivation and the like of different fluid-filled components of the device. For example, flow of fluid may be directed to an inflatable member to place the inflatable member in an inflated configuration. Similarly, flow of fluid may be directed away from the inflatable member to place the inflatable member in a deflated configuration. In some implantable fluid-operated devices, an implantable pumping device may be electrically operated. For example, in some devices, the implantable pumping device may be electrically operated using a device located outside of the body of the patient. Additionally, some such devices include antennas that allow for charging of the bodily implant and/or communication or control of the bodily implant from a device located outside of the body of the patient.

There is a need for bodily implant that efficiently incorporates a housing that contains components, such as electrical components, and antennas.

### SUMMARY

According to a general aspect, a medical device includes a bodily implant having a housing defining a cavity. The housing has a top portion defining a first opening, a second opening, and a third opening. The device includes an electronic control system that is at least partially disposed within the cavity. The device includes a fluid control system having a pump. The fluid control system is at least partially disposed within the cavity. The device includes a recharge antenna having a first portion extending through the first opening of the top portion of the housing and into the cavity defined by the housing. The recharge antenna has a second portion extending through the second opening of the top portion of the housing and into the cavity defined by the housing. The device includes a communications antenna extending through the third opening of the top portion of the housing and into the cavity defined by the housing.

In some implementations, the bodily implant includes an inflatable member configured to be placed in an inflated configuration and a deflated configuration.

In some implementations, the bodily implant includes a fluid reservoir and an inflatable member. In some implementations, the bodily implant includes a fluid reservoir operatively coupled to the housing, the bodily implant includes an inflatable member operatively coupled to the housing. In some implementations, the bodily implant includes a fluid reservoir fluidically coupled to the housing via a tubing member. In some implementations, the bodily implant includes an inflatable member fluidically coupled to the housing via a tubing member. In some implementations, the housing of the bodily implant includes a bottom portion, a first side portion, and a second side portion, the top portion, the bottom portion, the first side portion, and the second side portion collectively defining at least a portion of the cavity of the housing.

In some implementations, the housing of the bodily implant includes a bottom portion, a first side portion, and a second side portion, the top portion, the bottom portion, the first side portion, and the second side portion collectively defining at least a portion of the cavity of the housing, the top portion, the bottom portion, the first side portion, and the second side portion being unitarily formed. In some implementations, the housing of the bodily implant includes a bottom portion, a first side portion, and a second side portion, the top portion, the bottom portion, the first side portion, and the second side portion collectively defining at least a portion of the cavity of the housing, the top portion, the bottom portion, the first side portion, and the second side portion being unitarily formed of a metal material.

In some implementations, the housing of the bodily implant includes a bottom portion, a first side portion, a second side portion, a front portion, and a rear portion, the top portion, the bottom portion, the first side portion, the second side portion, the front portion, and the rear portion collectively defining at least a portion of the cavity of the housing.

In some implementations, the electronic control system includes a power storage device, the power storage device being at least partially disposed within the cavity defined by the housing. In some implementations, the fluid control system includes a plurality of valves.

In some implementations, the pump is a first pump, the fluid control system includes a second pump. In some implementations, the bodily implant is a penile implant. In some implementations, the bodily implant is an artificial sphincter.

According to an aspect, a medical device, includes a bodily implant having a housing, the housing includes a top portion, a first side portion, a second side portion, and a bottom portion collectively defining a cavity, the top portion of the housing defining a first opening, a second opening, and a third opening; a recharge antenna having a first portion extending through the first opening of the top portion of the housing and into the cavity, the recharge antenna having a second portion extending through the second opening of the top portion of the housing and into the cavity; and a communications antenna extending through the third opening of the top portion of the housing and into the cavity.

In some implementations, the top portion, the first side portion, the second side portion, and the bottom portion are unitarily formed. In some implementations, the bodily implant includes an inflatable member configured to be placed in an inflated configuration and a deflated configuration. In some implementations, the bodily implant includes a fluid reservoir fluidically coupled to the housing via a tubing member. In some implementations, the bodily implant includes an inflatable member fluidically coupled to the housing via a tubing member.

### BRIEF DESCRIPTION OF THE DRAWINGS

FIG. 1 is a block diagram of an implantable fluid-operated inflatable device.

FIG. 2A illustrates a system including an example implantable fluid-operated inflatable device.

FIG. 2B illustrates a system including an example implantable fluid-operated inflatable device.

FIG. 3 is a schematic diagram of a fluidic architecture of an implantable fluid-operated inflatable device.

FIG. 4 is an exploded view of a portion of an example implantable fluid-operated inflatable device.

FIG. 5 is a perspective view of a portion of a housing of the implantable fluid-operated inflatable device of FIG. 4.

FIG. 6 is a top view of a housing a of an example implantable fluid-operated inflatable device.

FIG. 7 is a front view of a housing of an example implantable fluid-operated inflatable device.

FIG. 8 is a perspective view of a modular feedthrough member.

FIG. 9 is a flow chart of a method 600 according to an implementation.

### DETAILED DESCRIPTION

Detailed implementations are disclosed herein. However, it is understood that the disclosed implementations are merely examples, which may be embodied in various forms. Therefore, specific structural and functional details disclosed herein are not to be interpreted as limiting, but merely as a basis for the claims and as a representative basis for teaching one skilled in the art to variously employ the implementations in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having,” as used herein, are defined as comprising (i.e., open transition). The term “coupled” or “moveably coupled,” as used herein, is defined as connected, although not necessarily directly and mechanically.

In general, the implementations are directed to bodily implants. The term patient or user may hereinafter be used for a person who benefits from the medical device or the methods disclosed in the present disclosure. For example, the patient can be a person whose body is implanted with the medical device or the method disclosed for operating the medical device by the present disclosure. The term physician may hereinafter be used for a person who places or implants a device within the body of the patient or otherwise provides instructions or counsel to the patient on how to use the device disposed within the body of the patient.

An implantable fluid-operated inflatable device may include a fluid control system. In some examples, the fluid control system includes at least one pump and/or at least one valve. In some examples, the components of the fluid control system control the flow of fluid between a fluid reservoir and an inflatable member of the implantable fluid-operated inflatable device, to provide for the inflation/pressurization and deflation/depressurization of the inflatable member. In some implementations, the fluid control system can be electronically operated.

For example, the pumps and/or valves of the fluid control system can be electronically operated by the fluid control system to control the pressure of, and the flow of fluid in, parts of the fluid-operated inflatable device. An electronically-operated fluid control system, in accordance with implementations described herein, can include a plurality of electromechanical devices, such as, piezoelectric devices that operate as pumps or as valves in the system. One or more processors or controllers can control the electromechanical devices.

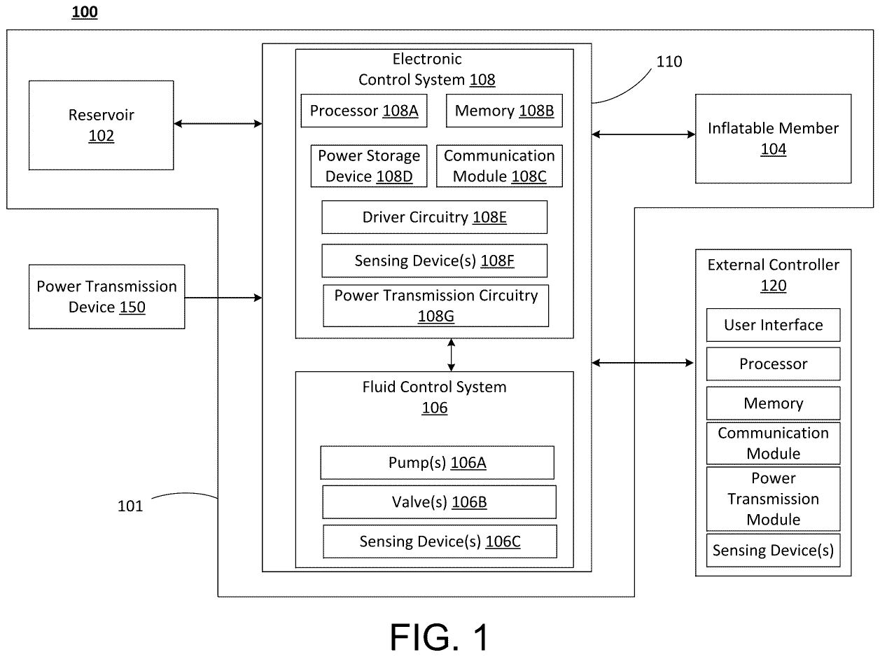

FIG. 1 is a block diagram of an example implantable fluid-operated inflatable device 100. The device 100 includes components that are configured to be implanted or disposed within a body of a patient or user and components that are configured to operate and be disposed outside of the body of the patient or user. The implantable portion 101 (the portion that is configured to be disposed within the body of the patient) includes a reservoir 102, an electronic control system 108, a fluid control system 106, and an inflatable member 104. The portions that are configured to be disposed outside of the body of the patient include a power transmission device 150 and an external controller 120.

In the example inflatable device 100, the electronic control system 108 may interface with a fluid control system 106. The fluid control system 106 can include fluidics components such as one or more pumps 106A, one or more valves 106B and the like configured to transfer fluid between the fluid reservoir 102 and the inflatable member 104. The fluid control system 106 can include one or more sensing devices 106C, such as, for example, one or more pressure sensors, one or more flow rate sensors, etc., that sense conditions such as, for example, fluid pressure, fluid flow rate and the like within the fluidics architecture of the inflatable device 100. In some implementations, the electronic control system 108 includes components that provide for the monitoring and/or control of the operation of various fluidics components of the fluid control system 106 and/or communication with one or more sensing device(s) within the implantable fluid-operated inflatable device 100 and/or communication with one or more external device(s). In some examples, the electronic control system 108 includes components such as a processor 108A, a memory 108B, a communication module 108C, a power storage device 108D (e.g., a battery), electronic driver circuity 108E, sensing devices 108F, such as, for example, voltage measurement circuitry, current measurement circuitry, an accelerometer, and other such components configured to provide for the monitoring, operation, and control of the implantable fluid-operated inflatable device 100, and power transmission circuitry 108G. In some examples, the communication module 108C of the electronic control system 108 may provide for communication with one or more external devices such as, for example, the external controller 120.

In some examples, the external controller 120 includes components such as, for example, a user interface, a processor, a memory, a communications module, a power transmission module, and other such components providing for operation and control of the external controller 120 and communication with the electronic control system 108 of the inflatable device 100. For example, the memory may store instructions, applications and the like that are executable by the processor of the external controller 120. The external controller 120 may be configured to receive user inputs via, for example, the user interface, and to transmit the user inputs, for example, via the communication module, to the electronic control system 108 for processing, operation, and control of the inflatable device 100. Similarly, the electronic control system 108 may, via the respective communication modules, transmit operational information to the external controller 120. This may allow operational status of the inflatable device 100 to be provided, for example, through the user interface of the external controller 120, to the user, may allow diagnostics information to be provided to a physician, a technician, and the like. In some implementations, the communications modules are configured to communicate via Bluetooth. In other implementations, the communication modules are configured to communicate via a different method such as via WiFi.

In some examples, the power transmission module of the external controller 120 provides for charging of the components of the internal electronic control system 108. In some examples, transmission of power for the charging of the internal electronic control system 108 can be, alternatively or additionally, provided by an external power transmission device 150 that is separate from the external controller 120. In some implementations the external controller 120 can include sensing devices such as one or more pressure sensors, one or more accelerometers, and other such sensing devices. In some implementations, a pressure sensor in the external controller 120 may provide, for example, a local atmospheric or working pressure to the internal electronic control system 108, to allow the inflatable device 100 to compensate for variations in pressure. In some implementations, an accelerometer in the external controller 120 may provide detected patient movement to the internal electronic control system 108 for control of the inflatable device 100.

The fluid reservoir 102, the inflatable member 104, the electronic control system 108 and the fluid control system 106 may be internally implanted into the body of the patient. In some implementations, the electronic control system 108 and the fluid control system 106 are coupled in, or incorporated into, a housing 110. In some implementations, at least a portion of the electronic control system 108 is physically separate from the fluid control system 106. In some implementations, some modules of the electronic control system 108 are coupled to, or incorporated into, the fluid control system 106, and some modules of the electronic control system 108 are separate from the fluid control system 106. For example, in some implementations, some modules of the electronic control system 108 are included in an external device (such as the external controller 120) that is in communication other modules of the electronic control system 108 included within the implantable fluid-operated inflatable device 100.

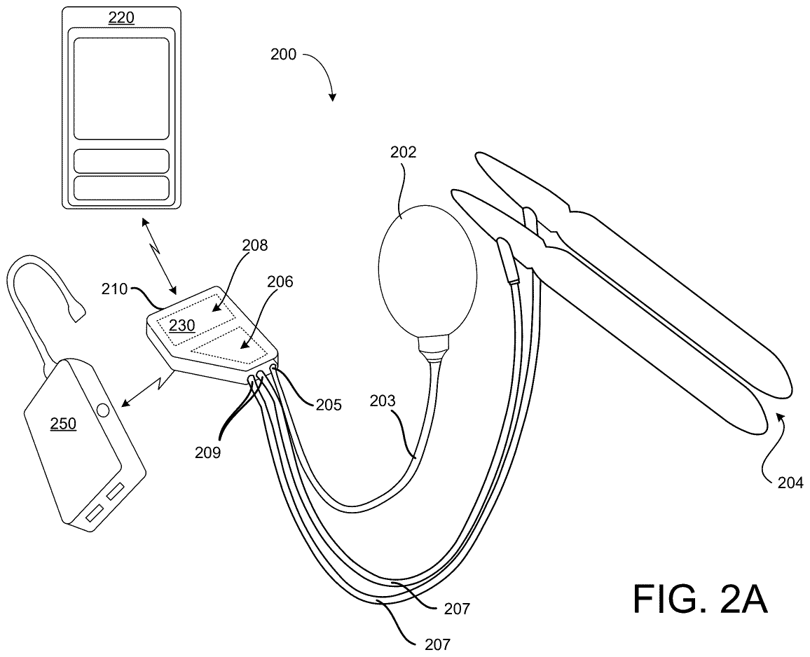

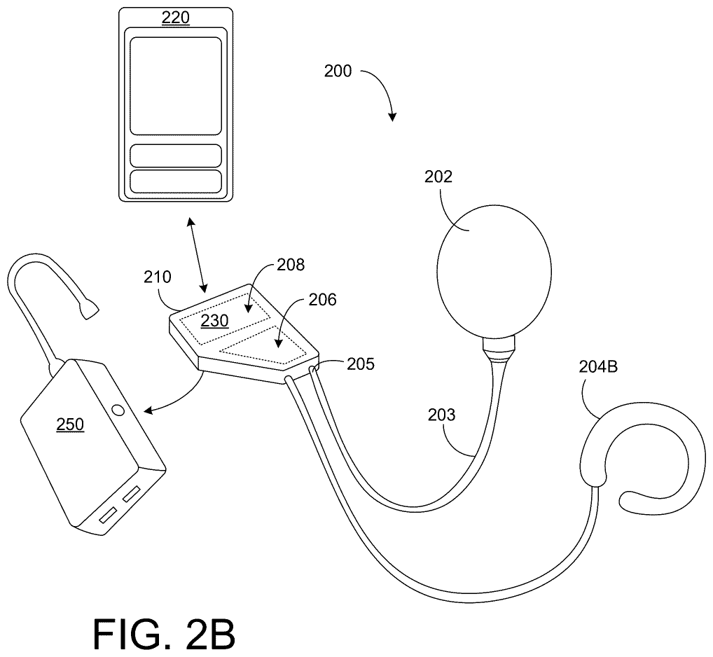

The example implantable fluid-operated inflatable device 100 may be representative of a number of different types of implantable fluid-operated devices. For example, the implantable fluid-operated inflatable device 100 shown in FIG. 1 may be representative of an inflatable penile prosthesis as shown in FIG. 2A. In another example, the implantable fluid-operated inflatable device 100 as shown in FIG. 2B may be representative of an artificial sphincter (such as an artificial urinary sphincter) that includes an inflatable member 204B that forms a loop or cuff. In some implementations, the example implantable fluid-operated inflatable device 100 shown in FIG. 1 may be representative of other types of implantable inflatable devices that rely on the control of fluid flow to components of the device to achieve inflation, pressurization, deflation, depressurization, deactivation, and the like.

An example system including an example implantable fluid-operated inflatable device 200 in the form of an example inflatable penile prosthesis is shown in FIG. 2A. The example inflatable device 200 includes a fluid control system 206 (similar to the example fluid control system 106 described above with respect to FIG. 1) including fluidics components such as pumps, valves, sensing devices and the like positioned in fluid passageways. In some implementations, the fluid control system includes components such as, for example, one or more fluid control devices, one or more pressure sensors, and other such components. In some implementations, the example inflatable device 200 includes an electronic control system 208 (similar to the example electronic control system 108 described above with respect to FIG. 1) configured to provide for the transfer of fluid between a reservoir 202 (such as the example fluid reservoir 102 described above with respect to FIG. 1) and an inflatable member 204 (similar to the example inflatable member 104 described above with respect to FIG. 1) via the fluidics components. In the example shown in FIG. 2A, the inflatable member 204 is in the form of a pair of inflatable cylinders. In the example shown in FIG. 2A, fluidics components of the fluid control system 206, and electronic components of the electronic control system 208 are received in a housing 210. In some implementations, fluidics components of the fluid control system 206, and electronic components of the electronic control system 208 received in the housing 210 together define an electronically controlled fluid manifold 230 that provides for the electronic control of the flow of fluid between the reservoir 202 and the inflatable member 204.

In the examples shown in FIG. 2A and FIG. 2B, a first conduit 203 connects a first fluid port 205 of the electronically controlled fluid manifold 230 (the fluid control system 206/electronic control system 208 received in the housing 210) with the reservoir 202. One or more second conduits 207 connect one or more second fluid ports 209 of the electronically controlled fluid manifold 230 (the fluid control system 206/electronic control system 208 received in the housing 210) with the inflatable member 204 in the form of the inflatable cylinders. In some examples, the electronic control system 208 can communicate with an external controller 220 (similar to the external controller 120 described above with respect to FIG. 1), via respective communication modules.

In some implementations, an application stored in a memory and executed by a processor of the external controller 220 may allow the patient or user to operate, view, monitor and alter operation of the inflatable device 200. For example, the patient or user may be able to use the external controller 220 to cause the inflatable device 200 (or the inflatable member) to be placed in its inflated configuration or may be able to cause the inflatable device 200 (or the inflatable member) to be placed in its deflated configuration.

In some examples, components of the electronic control system 208 and/or the fluid control system 206 can be charged and/or recharged by a power transmission module of the external controller 220, and/or by a power transmission device 250, that is separate from the external controller 220.

The principles to be described herein are applicable to the example implantable fluid-operated inflatable device, in the form of the example inflatable penile prostheses shown in FIG. 2A, and to other types of implantable fluid-operated inflatable devices that rely on pumps, valves and/or various fluidics components to provide for the transfer of fluid between the different fluid-filled implantable components to achieve inflation, deflation, pressurization, depressurization, deactivation, occlusion, and the like for effective operation. The example implantable fluid-operated inflatable device 200 shown in FIG. 2A includes an electronic control system 208 to provide for control of the operation of the respective inflatable members 204 in the form of cylinders, and the monitoring and control of pressure and/or fluid flow through inflatable members 204. Some of the principles to be described herein may also be applied to implantable fluid-operated inflatable devices that are manually controlled.

As noted above, the electronic control system 208 controlling the flow of fluid between the reservoir 202 and the inflatable member 204 for inflation, pressurization, deflation, depressurization and the like of the inflatable member 204 may provide for improved patient control and physician control of the inflatable device 200, improved accuracy in operation of the inflatable device 200, improved patient comfort, improved patient safety, and the like. In some situations, this improved control and improved accuracy in the operation of the inflatable device 200 may rely on precise operation and control of the components within the fluid control system 206 and/or the electronically controlled fluid manifold 230. Accordingly, in some implementations, the electronically controlled fluid manifold 230 includes a fluid control system 206 having one or more pump and one or more valve devices and one or more sensing devices. Accurate and consistent operation of the components of the pump and/or valve devices may produce the desired accurate flow control, and consistent inflation, deflation, pressurization, depressurization, deactivation, occlusion, and the like for effective operation.

A fluid control system, in accordance with implementations described herein, can include a pump assembly including, for example, one or more pump devices and valve devices within a fluid circuit of the pump assembly to control the transfer fluid between the fluid reservoir and the inflatable member. In some examples, the pump assembly including the one or more pump devices and valve device(s) is electronically controlled. In an example in which the pump assembly is electronically powered and/or controlled, the pump assembly may include a hermetic manifold that can contain and segment the flow of fluid from electronic components of the pump assembly, to prevent leakage and/or gas exchange. In some examples, the one or more pump devices and valve devices include electric elements that are configured to be electronically actuated to change their shape and thereby to function as a pump or valve. In some examples, the pump assembly includes one or more pressure sensing devices in the fluid circuit to provide for relatively precise monitoring and control of fluid flow and/or fluid pressure within the fluid circuit and/or the inflatable member. A fluid circuit configured in this manner may facilitate the proper inflation, deflation, pressurization, depressurization, and deactivation of the components of the implantable fluid-operated device to provide for patient safety and device efficacy.

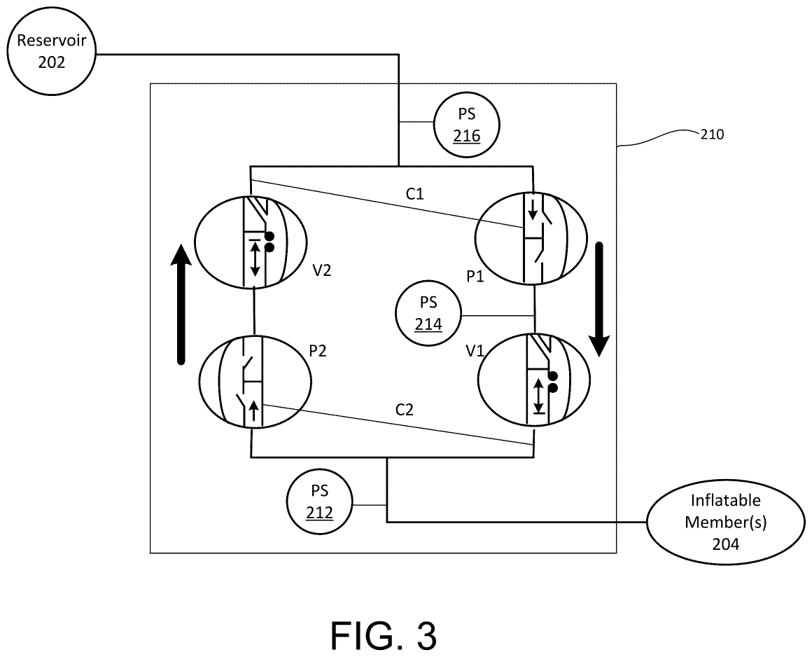

FIG. 3 is a schematic diagram of an example fluidic architecture for an electronically-operated implantable fluid-operated inflatable device, according to an aspect. The fluidic architecture of an implantable fluid-operated inflatable device can include other arrangements of fluidic passageways, pump(s)/valve(s), pressure sensor(s) and other components than the examples shown in FIG. 3.

The example fluidic architecture shown in FIG. 3 includes a first pump P1 and a first valve V1 positioned in a first fluid passageway, between the reservoir 202 and the inflatable member 204, to control the flow of fluid from the reservoir 202 to the inflatable member 204. The example fluidic architecture shown in FIG. 3 includes a second pump P2 and a second valve V2 positioned in a second fluid passageway, between the inflatable member 204 and the reservoir 202, to control the flow of fluid from the inflatable member 204 to the reservoir 202.

In example fluidic architecture shown in FIG. 3, the first pump P1 and the first valve V1 operate to pump fluid from the reservoir 202 to the inflatable member 204 through the first fluid passageway to provide for inflation of the inflatable member 204, while the second valve V2 closes the second fluid passageway to prevent backflow of fluid, back to the reservoir 202. The second pump P2 and the second valve V2 operate to pump fluid from the inflatable member 204 to the reservoir 202 through the second fluid passageway to provide for deflation of the inflatable member 204, while the first valve V1 closes the first fluid passageway to prevent backflow of fluid to the inflatable member 204.

In an example implementation, a conduit C1 can connect a section of the second fluid passageway that is downstream of pump P2 and valve V2 to a section of the first fluid passageway, for example, to an inlet portion of pump P1. Fluid flow through conduit C1 can flush fluid and material out from of the section of the first fluid passageway when fluid is pumped from the inflatable member 204 to the reservoir 202. In an example implementation, a conduit C2 can connect a section of the first fluid passageway that is downstream of pump P1 and valve V1 to a section of the second fluid passageway, for example, to an inlet portion of pump P2. Fluid flow through conduit C2 can flush fluid and material out from of the section of the second fluid passageway when fluid is pumped from the reservoir 202 to the inflatable member 204.

In some implementations, the example fluidic architecture can include one or more pressure sensors 212, 214, 216, each configured to measure a fluid pressure at a point in the system. For example, a first pressure sensor 212 can be connected to a fluidic passageway, conduit, chamber or component located fluidically between the inflatable member 204 and pumps P1, P2 and valves V1, V2, and can be configured to measure a fluid pressure at this location, which can also serve as a measure of a fluid pressure in the inflatable member(s) 204, because the fluid is essentially incompressible and the conduit between the pressure sensor 212 and the inflatable member(s) 204 can be considered to be free of obstruction. A second pressure sensor 214 can be connected to a fluidic passageway, conduit, chamber or component located fluidically between pump P1 and valve V1 and can be configured to measure a fluid pressure at this location. A third pressure sensor 216 can be connected to a fluidic passageway, conduit, chamber or component located fluidically between the reservoir 202 and pumps P1, P2 and valves V1, V2, and can be configured to measure a fluid pressure at this location, which can also serve as a measure of a fluid pressure in the reservoir, because the fluid is essentially incompressible and the conduit between the pressure sensor 216 and the reservoir 202 can be considered to be free of obstruction. In some implementations one or more of the pressure sensors 212, 214, 216 can be contained with the housing 210.

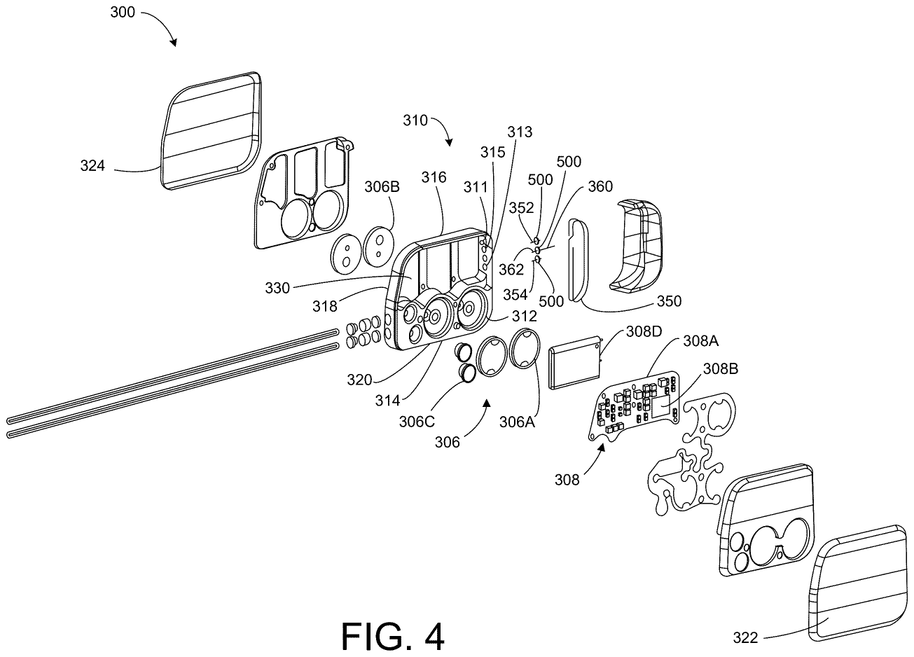

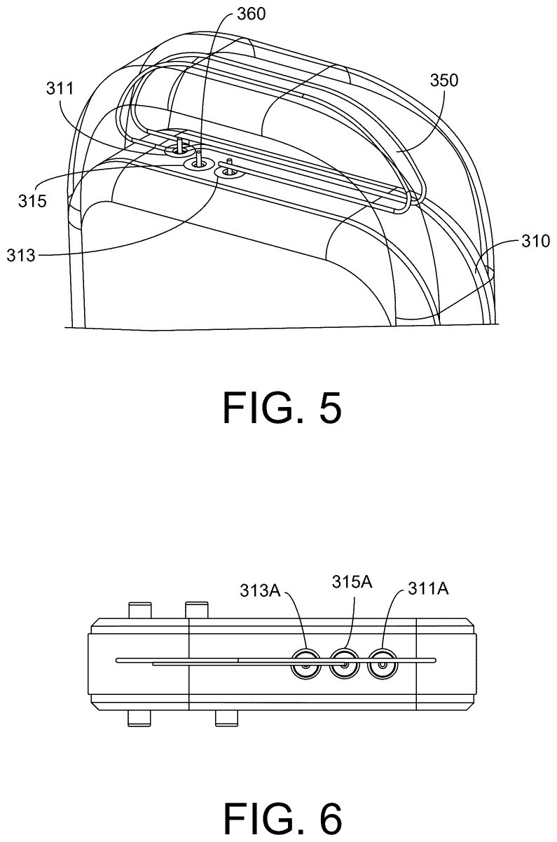

FIG. 4 is an exploded view of a portion of an example implantable fluid-operated inflatable device 300. FIG. 5 is a perspective view of a portion of a housing 310 of the implantable fluid-operated inflatable device 300. The housing 310 includes a top portion 312, a first side portion 314, a second side portion 316, and a bottom portion 318. The top portion 312, the first side portion 314, the second side portion 316, and the bottom portion 318 collectively define a cavity 330. In the illustrated implementation, the housing 310 also includes a first edge member or front portion 322 and a second edge member or rear portion 324. The first edge member or front portion 322 and the second edge member or rear portion 324 also along with the top portion 312, the first side portion 314, the second side portion 316, and the bottom portion 318 define the cavity 330.

In the illustrated implementation, the housing 310 is formed of three individual pieces or members. Specifically, the housing 310 is formed of the center member 320, the first edge member 322, and the second edge member 324. The center member 320 includes the top portion 312, the first side portion 314, the second side portion 316, and the bottom portion 318. In the illustrated implementation, the center member 320 is unitarily formed (or is monolithic or molded or formed of a single piece of material). In the illustrated implementation, the first edge member 322 and the second edge member 324 are also each unitarily formed. In other implementations, any or all of the center member 320, the first edge member 322, and the second edge member 324 may be formed of more than one piece or members. In some implementations, the center member 320, the first edge member 322, and the second edge member 324 are formed of a metal material. For example, in some implementations, the center member 320, the first edge member 322, and the second edge member 324 are formed of titanium. In other implementations, such members are formed of a different type of metal material. In yet other implementations, such members are formed of a different material.

In some implementations, the center member, the first edge member, and the second edge member are coupled and hermetically sealed.

In the illustrated implementation, the housing 310 has a specific shape. In other implementations, the housing 310 has a shape other than as illustrated.

In the illustrated implementation, the cavity 330 of the housing 310 houses or contains at least a portion of an electronic system 308 of the device 300. For example, in the illustrated implementation, the processor 308A, the memory 308B, and the power storage device 308D are disposed within the cavity 330. In other implementations, other components of an electrical system are disposed within or at least partially disposed within the cavity 330.

In the illustrated implementation, the cavity 330 of the housing 310 houses or contains at least a portion of a fluid control system 306 of the device 300. For example, in the illustrated implementation, the pumps 306B, the valves 306A, and the sensing devices 306C are disposed within the cavity 330. In other implementations, other components of a fluid control system are disposed within or at least partially disposed within the cavity 330.

In the illustrated implementation, the housing 310 is operatively coupled to a reservoir (such as reservoir 102 or 202). Specifically, housing 310 is coupled to the reservoir via a tubular member 370. Similarly, the housing 310 is also operatively coupled to an inflatable member (such as inflatable member 104 or 204). The housing 310 is coupled to the inflatable member via a tubular member 372.

In the illustrated implementation, the device 300 includes a first antenna 350 and a second antenna 360. The first antenna 350 may be a recharge antenna that is configured to interact with an external power transmission device such as power transmission device 150. In some implementations, the power storage device 308D may be recharged via the recharge antenna 350. The second antenna 360 may be a communications antenna that is configured to interact with an external control device such as external controller 120. In some implementations, the device 300 may be controlled from outside of a body of a patient via the communications antenna and the instructions or communications received by the communications antenna from the external control device.

In the illustrated implementation, the first antenna 350 and the second antenna 360 extend from the housing 310. Specifically, the first antenna 350 includes a first portion 352 that extends through a first opening 311 defined by the top portion 312 of the housing 310. The first antenna 350 also includes a second portion 354 that extends through a second opening 313 defined by the top portion 312 of the housing 310. The portions of the first antenna 350 that extend into the cavity 330 of the housing 310 may be operatively coupled to power transmission circuitry (such as power transmission circuitry 108G) or other circuitry to harvest power received by the first antenna 350 to thereby charge the power storage device 108D.

In the illustrated implementation, the second antenna 360 includes a first portion 362 that extends through a third opening 315 defined by the top portion 312 of the housing 310. The portion of the second antenna 360 that extends into the cavity 330 of the housing 310 may be operatively coupled to a communications module (such as communication module 108C) to provide communications received from outside of the body of the patient to the device 300.

In the illustrated implementation, the portion of the first antenna 350 and the portion of the second antenna 360 that are disposed outside of the cavity 330 of the housing 310 are disposed within a material. For example, the material may be a transparent material such as silicone or a plastic material.

In the illustrated implementation, modular feed-throughs 500 (described in more detail below) are used to facilitate the extension of the antennas 350 and 360 through the openings 311, 313, and 315.

As best illustrated in FIG. 5, the openings 311, 313, and 315 defined by the top portion 312 of the housing 310 are offset from each other. In other words, the openings 311, 313, and 315 are not disposed along a straight line. Additionally, in the implementations, the openings are set towards one side of the top portion 312 of the housing 310. In other implementations, the opening may be arranged differently or may be disposed on different portions of the top portion or on different portions of the housing.

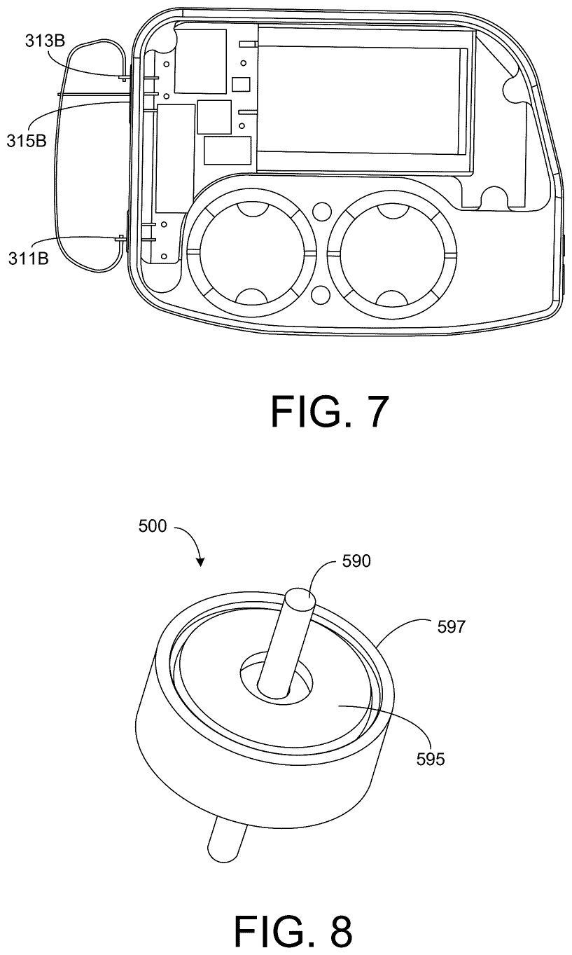

As illustrated in FIG. 6, in another implementation, the openings 311A, 313A, and 315A are aligned. In other words, the openings 311A, 313A, and 315A are disposed in or along a line. As illustrated in FIG. 7, the openings 311B, 313B, and 315B are disposed apart from each other. More specifically, one opening is disposed towards one end of the top portion and another opening is disposed towards another end of the top portion.

FIG. 8 is a perspective view of a modular feed-through 500. The modular feed-through 500 may be made and then coupled to the housing. For example, the modular feed-through 500 may be coupled to the top portion of the housing within one of the openings (such as openings 311, 313, and 315) defined by the top portion of the housing.

In the illustrated implementation, the modular feed-through 500 includes a wire 590 formed from a metal material. The wire 590 may be considered a portion of the antennas (such as antennas 350 and 360) and may be coupled to other portions of the antennas. For example, in some implementations, the wire is formed of a low resistivity metal or a malleable metal such as platinum iridium. In other implementations, the wire 590 is formed of a different metal material. The wire 590 may be considered a portion of the antennas (such as antennas 350 and 360) and may be coupled to other portions of the antennas.

In the illustrated implementation, the modular feed-through 500 includes a middle portion 595 that surrounds the wire 590 and an outer portion 597 that surrounds the middle portion 595. The middle portion 595 is disposed between the wire 590 and the outer portion 597. The middle portion 595 may be formed of a non-conductive material. In some implementations, the middle portion 595 is formed of ceramic, alumina, or glass. In other implementations, the middle portion 595 is formed of a different non-conductive material.

The outer portion 597 may be formed of any material that may be used to form a hermetic seal with the housing. For example, in some implementations, the outer portion 597 is formed of a metal material such as titanium. In some implementations, the outer portion is formed of the same material as the housing.

In some implementations, the wire 590 is coupled to the middle portion 595 via a brazing process using either gold or another active brazing alloy. Similarly, in some implementations, the middle portion 595 is coupled to the outer portion 597 via a brazing process using either gold or another active brazing alloy. In some implementations, the feedthrough 500 is brazed with the gold (or other active brazing alloy) joints facing up so gravity and capillary actions draw the gold (or other active brazing alloy) into the space between the wire 590 and the middle portion 595 and between the middle portion 595 and the outer portion 597 to create hermetic joints or seals.

In the illustrated implementation, the modular feed-through has a circular cross-section. In some cases, the circular cross-section may facilitate the manufacture and also facilitate the coupling to the top portion of the housing. For example, the circular cross-section may facilitate coupling the modular feed-through to the top portion of the housing while forming a hermetic seal.

In some implementations, as illustrated in FIG. 4, the antennas 350 and 360 may be coupled to (such as welded to) one end of the wire 590 of the modular feed-through. In such examples, the wire 590 becomes or forms part of the antenna which extends into the cavity 330 of the housing. In some implementations, the wire 590 and the antenna may be unitarily or monolithically formed (rather than being two pieces that are coupled together).

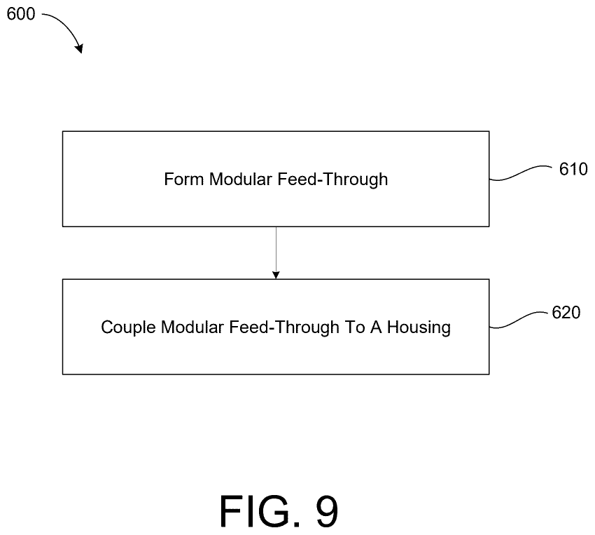

FIG. 9 is a flow chart of a method 600 according to an implementation. At 610 a modular feed-through is formed. In some implementations, the modular feed-through may be formed according to the processes described above. At 620 the modular feed-though is coupled to a housing. In some implementations, the coupling of the modular feed-through forms a hermetic joint or seal. In some implementations, the modular feed-through is welded, such as through a laser welding process, to the housing. In other implementations, the modular feed-through is coupled to the housing via a brazing process. In other implementations, a different method of coupling is used.

While certain features of the described implementations have been illustrated as described herein, many modifications, substitutions, changes and equivalents will now occur to those skilled in the art. It is, therefore, to be understood that the will and in and in appended claims are intended to cover all such modifications and changes as fall within the scope of the embodiments.

What is claimed is:

1. A medical device, comprising:
a bodily implant having a housing defining a cavity, the housing having a top portion defining a first opening, a second opening, and a third opening;
an electronic control system at least partially disposed within the cavity;
a fluid control system having a pump, the fluid control system being at least partially disposed within the cavity;
a recharge antenna having a first portion extending through the first opening of the top portion of the housing and into the cavity defined by the housing, the recharge antenna having a second portion extending through the second opening of the top portion of the housing and into the cavity defined by the housing; and
a communications antenna extending through the third opening of the top portion of the housing and into the cavity defined by the housing.

2. The medical device of claim 1, wherein the bodily implant includes an inflatable member configured to be placed in an inflated configuration and a deflated configuration.

3. The medical device of claim 1, wherein the bodily implant includes a fluid reservoir and an inflatable member.

4. The medical device of claim 1, wherein the bodily implant includes a fluid reservoir operatively coupled to the housing, and the bodily implant includes an inflatable member operatively coupled to the housing.

5. The medical device of claim 1, wherein the bodily implant includes a fluid reservoir fluidically coupled to the housing via a tubing member.

6. The medical device of claim 1, wherein the bodily implant includes an inflatable member fluidically coupled to the housing via a tubing member.

7. The medical device of claim 1, wherein the housing of the bodily implant includes a bottom portion, a first side portion, and a second side portion; the top portion, the bottom portion, the first side portion, and the second side portion collectively define at least a portion of the cavity of the housing.

8. The medical device of claim 1, wherein the housing of the bodily implant includes a bottom portion, a first side portion, and a second side portion, the top portion, the bottom portion, the first side portion, and the second side portion collectively define at least a portion of the cavity of the housing; and the top portion, the bottom portion, the first side portion, and the second side portion being unitarily formed.

9. The medical device of claim 1, wherein the housing of the bodily implant includes a bottom portion, a first side portion, and a second side portion; the top portion, the bottom portion, the first side portion, and the second side portion collectively define at least a portion of the cavity of the housing; and the top portion, the bottom portion, the first side portion, and the second side portion being unitarily formed of a metal material.

10. The medical device of claim 1, wherein the housing of the bodily implant includes a bottom portion, a first side portion, a second side portion, a front portion, and a rear portion; the top portion, the bottom portion, the first side portion, the second side portion, the front portion, and the rear portion collectively defining at least a portion of the cavity of the housing.

11. The medical device of claim 1, wherein the electronic control system includes a power storage device, the power storage device being at least partially disposed within the cavity defined by the housing.

12. The medical device of claim 1, wherein the fluid control system includes a plurality of valves.

13. The medical device of claim 1, wherein the pump is a first pump, and the fluid control system includes a second pump.

14. The medical device of claim 1, wherein the bodily implant is a penile implant.

15. The medical device of claim 1, wherein the bodily implant is an artificial sphincter.

16. A medical device, comprising:
a bodily implant having a housing, wherein the housing includes a top portion, a first side portion, a second side portion, and a bottom portion collectively defining a cavity, the top portion of the housing defining a first opening, a second opening, and a third opening;
a recharge antenna having a first portion extending through the first opening of the top portion of the housing and into the cavity, the recharge antenna having a second portion extending through the second opening of the top portion of the housing and into the cavity; and
a communications antenna extending through the third opening of the top portion of the housing and into the cavity.

17. The medical device of claim 16, wherein the top portion, the first side portion, the second side portion, and the bottom portion are unitarily formed.

18. The medical device of claim 16, wherein the bodily implant includes an inflatable member configured to be placed in an inflated configuration and a deflated configuration.

19. The medical device of claim 16, wherein the bodily implant includes a fluid reservoir fluidically coupled to the housing via a tubing member.

20. The medical device of claim 16, wherein the bodily implant includes an inflatable member fluidically coupled to the housing via a tubing member.
